(19) United States
(12) Patent Application Publication
(10) Pub. No.: US 2020/0038399 A1
(43) Pub. Date: Feb. 6, 2020

(54) FORMULATIONS, METHODS, KIT, AND DOSAGE FORMS FOR TREATING BACTERIAL INFECTION

(71) Applicant: Motif BioSciences Inc., New York, NY (US)

(72) Inventors: David Huang, Houston, TX (US); Sergio Lociuro, Rancate (CH); Khalid Islam, Lugano (CH); Keith Bostian, Short Hills, NJ (US)

(21) Appl. No.: 16/597,975

(22) Filed: Oct. 10, 2019

Related U.S. Application Data
(63) Continuation of application No. 15/800,960, filed on Nov. 1, 2017.
(60) Provisional application No. 62/420,634, filed on Nov. 11, 2016.

(51) Int. Cl. A61K 31/506; A61K 31/42; A61K 9/20

(52) U.S. Cl. CPC A61K 31/506; A61K 31/42; A61K 9/0019; A61K 9/2059; A61K 2300/00; A61K 9/2013

(57) ABSTRACT

Pharmaceutical formulations for treating bacterial or fungal infections comprising iclaprim or its enantiomers and a sulfonamide antibiotic, and treatment and manufacturing methods, kits and dosage forms thereof, are provided.

### TECHNICAL FIELD

Embodiments of the disclosure relate generally to formulations, methods, kits, and dosage forms for treating bacterial infection.

### BACKGROUND

Racemic iclaprim (MTF-100, which is also known as AR-100) is a potent inhibitor of microbial dihydrofolate reductase (DHFR) that is used to treat bacterial infections such as acute bacterial skin, skin structure infections (ABSSSI) or hospital-acquired bacterial pneumonia (HABP). Iclaprim is a broad-spectrum bactericidal antibiotic which has a low propensity for resistance development. Iclaprim also exhibits an alternative mechanism of action against bacterial pathogens, including Gram-positive isolates of many staphylococcal, streptococcal, and enterococcal genera, as well as various Gram-positive pathogens that are resistant to antibiotic treatment; e.g., methicillin-resistant Staphylococcus aureus (MRSA). The two iclaprim enantiomers also show antibiotic activity, although some differences in pharmacokinetics and toxicity have been observed between them. Racemic iclaprim and its enantiomers thus have the potential to be an effective drug for treating infections of bacteria and fungi that have become resistant to standard antibiotics.

Sulfamethoxazole is a sulfonamide antibiotic used for treatment of both Gram negative and Gram positive bacterial infections. Due to the known side effects of the sulfonamides and their propensity for inducing drug-resistance in bacteria, sulfamethoxazole is no longer used alone but is administered in combination with trimethoprim. This combination is sold under the trade name Bactrim™.

Bactrim™ is used to treat urinary tract infections, acute otitis media, bronchitis, Shigellosis, traveler's diarrhea, methicillin-resistant MRSA and other bacterial infections, as well as certain fungal infections such as Pneumocystis pneumonia. However, the combination of sulfamethoxazole and trimethoprim can also cause severe side effects, such as loss of appetite, nausea, vomiting, painful or swollen tongue, dizziness or vertigo, tinnitus or insomnia.

Thus, there remains a need for effective pharmaceutical formulations for treating bacterial or fungal infections comprising sulfonamides and an additional antibiotic, which do not promote antibiotic resistance and which exhibit reduced side effects as compared to sulfonamides alone or sulfonamides in combination with trimethoprim, and methods, kits, and dosage forms thereof.

### SUMMARY

The present disclosure relates to pharmaceutical formulations, methods, kits, and dosage forms for treating bacterial infection. In one embodiment, the present disclosure provides pharmaceutical formulations comprising iclaprim and a sulfonamide antibiotic. In other embodiments, the iclaprim and sulfonamide antibiotic are present in the pharmaceutical formulations in a ratio of less than 1:5 of iclaprim to sulfonamide antibiotic. In one embodiment, the iclaprim and sulfonamide antibiotic are present in the pharmaceutical formulations in a ratio of from about 1:0.5 to 1:4 of iclaprim to sulfonamide antibiotic. In other embodiments, the pharmaceutical formulation can be formulated for intravascular (e.g., intravenous), intramuscular, inhalation, rectal, sublingual or oral administration, and can be provided in one or more dosage forms for such administration. The iclaprim comprising the pharmaceutical formulations can be the racemate, the R-enantiomer or the S-enantiomer of iclaprim. In one embodiment, the iclaprim comprising the pharmaceutical formulations is the R-enantiomer.

In other embodiments, the present disclosure provides methods of treating a bacterial infection in a subject, comprising administering to the subject a therapeutically effective amount of a pharmaceutical formulation comprising iclaprim or its enantiomers and a sulfonamide antibiotic.

In other embodiments, the present disclosure provides methods of manufacturing a pharmaceutical formulation for treating a bacterial infection in a subject, comprising combining iclaprim and a sulfonamide antibiotic. In still other embodiments, the present disclosure provides the use of iclaprim or its enantiomers and a sulfonamide antibiotic to manufacture a medicament for the treatment of a bacterial or fungal infection in a subject.

In other embodiments, the present disclosure provides kits comprising at least one dosage form comprising a pharmaceutical composition, wherein the pharmaceutical formulation comprises iclaprim or its enantiomers and a sulfonamide antibiotic, and optionally instructions for administering the at least one dosage form to treat bacterial infection in a subject.

### BRIEF DESCRIPTION OF THE FIGURES

FIG. 1 is a graph showing the concentration-in vitro activity relationships of a 1:5 Trimethoprim/Sulfamethoxazole combination (“TMP/SMX”) (solid square), Iclaprim (“AR-100”) (solid circle) and a 1:5 Iclaprim/Sulfamethoxazole combination (“AR-100/SMX”) (solid triangle).

### DETAILED DESCRIPTION

The following detailed description is exemplary and explanatory and is intended to provide further explanation of the present disclosure described herein. Other advantages and novel features will be readily apparent to those skilled in the art from the following detailed description of the present disclosure. U.S. Provisional Application Ser. No. 62/331,623 (filed on May 4, 2016), U.S. Provisional Application Ser. No. 62/469,781 (filed on Mar. 10, 2017), U.S. Provisional Application Ser. No. 62/420,634 (filed on Nov. 11, 2016), U.S. patent application Ser. No. 15/586,021 (filed on May 3, 2017) and U.S. patent application Ser. No. 15/586,815 (filed on May 4, 2017), are each incorporated herein in their entirety.

Bactrim™ is a synthetic antibacterial combination of sulfamethoxazole and trimethoprim which is used for treating various bacterial infections. It is available in tablets for oral administration, and can also be administered intravenously as a solution. A typical oral dose of Bactrim™ contains either 160 mg of trimethoprim and 800 mg of sulfamethoxazole or 80 mg of trimethoprim and 400 mg or sulfamethoxazole, and one or two doses can be given twice a day for up to 14 days. Intravenous Bactrim™ can be given in a total daily dose of 15 to 20 mg/kg (based on the trimethoprim component) in equally divided doses every 6 to 8 hours for up to 14 days. In either form, the typical ratio of trimethoprim and sulfamethoxazole is 1:5. In vitro studies have shown that bacterial resistance develops more slowly with both sulfamethoxazole and trimethoprim in combination than with either sulfamethoxazole or trimethoprim alone.

However, sulfonamides such as sulfamethoxazole show toxicity when given in higher doses, and care must be taken when administering this drug either alone or in combination with other antibiotics. For example, sulfamethoxazole can cause gastrointestinal disturbances such as nausea or vomiting; skin rashes, including Stevens-Johnson syndrome (aching joints and muscles; redness, blistering, and peeling of the skin); toxic epidermal necrolysis (difficulty in swallowing; peeling, redness, loosening, and blistering of the skin), liver damage; low white blood cell count; low platelet count (thrombocytopenia); agranulocytosis; aplastic anemia; and other blood disorders. Pyrexia, hematuria and crystalluria are also potential late manifestations of sulfamethoxazole overdoses. High doses of trimethoprim can also cause side effects, such as nausea, vomiting, dizziness, confusion or depression.

The present invention thus provides, in one embodiment, pharmaceutical formulations comprising a sulfonamide antibiotic with iclaprim instead of trimethoprim. The inventive pharmaceutical formulations are effective in treating bacterial infections, and exhibit less side effects than standard sulfonamide antibiotic combinations like Bactrim™. The inventors have surprisingly found a synergistic antibiotic effect when iclaprim is administered with a sulfonamide antibiotic in an iclaprim to sulfonamide antibiotic dose ratio that is lower than the 1:5 trimethoprim-sulfamethoxazole ratio expected to be most effective based on clinical and patient experience with Bactrim. This synergistic effect allows less sulfonamide and iclaprim antibiotic to be used, and thus the inventive pharmaceutical formulations are less toxic than standard sulfonamide antibiotic combinations.

The sulfonamide antibiotic used in the pharmaceutical formulations of the invention can comprise any known sulfonamide antibiotic, for example sulfamethiozole, sulfathiozole, sulfacarbamide, sulfathiourea, sulfadiazine, sulfisoxazole, sulfadimethoxine, sulfamethoxazole, 4-sulfanilamido-5,6-dimethoxy-pyrimidine (sulfadoxine), 2-sulfanilamido-4,5-dimethyl-pyrimidine, sulfaquinoxaline, sulfadiazine, sulfamonomethoxine, and 2-sulfanilamido-4,5-dimethyl-isoxazole or dapsone. In one embodiment, the sulfonamide antibiotic used in the pharmaceutical formulations of the invention comprises sulfamethoxazole. In preferred embodiments, the sulfonamide antibiotic used in the pharmaceutical formulations comprises sulfamethiozole or sulfathiozole. Combinations of different sulfonamide antibiotics may also be used.

Sulfamethoxazole, also known as N1-(5-methyl-3-isoxazolyl)sulfanilamide, has a molecular formula of C₁₀H₁₁N₃O₃S, and melting point range of 168-172° C. and a molecular weight of 253.28. Sulfamethoxazole is very slightly soluble in water, but is soluble 1 part in 50 parts alcohol. It is also soluble in alkali hydroxides. A 10% suspension in water has a pH of 4 to 6.4. Sulfamethoxazole has the following structural formula:

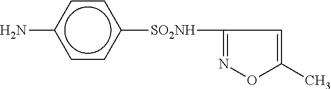

One skilled in the art can readily obtain sulfamethoxazole or synthesize this compound according to well-known methods.

Iclaprim, also known as 5-[(2RS)-2-cyclopropyl-7,8-dimethoxy-2Hchromen-5-ylmethyl] pyrimidine-2,4-diamine or 5-[[(2RS)-2-cyclopropyl-7,8-dimethoxy-2H-1-benzopyran-5-yl]methyl]pyrimidine-2,4-diamine, is racemic and is typically synthesized as the mesylate salt. The molecular formulae for iclaprim and iclaprim mesylate are C₁₉H₂₃N₄O₃ (base) and C₂₀H₂₆N₄O₆S (mesylate), and their relative molecular masses are 354.41 (base) or 450.52 (mesylate). General properties of iclaprim mesylate include, for example, a pH value of 4.2 for a 1% solution in water and a pKₐ of 7.2, a melting point range of 200−204° C., and solubility in water at 20° C. of approximately 10 mg/mL. The iclaprim mesylate salt has been formulated in a sterile aqueous/ethanolic vehicle as a concentrated solution for intravenous infusion after dilution for clinical testing on humans. The structural formula for iclaprim mesylate is:

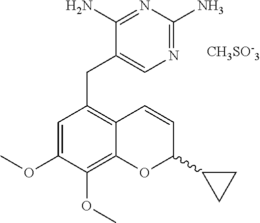

One skilled in the art can readily obtain iclaprim or iclaprim mesylate, and synthesis of these compounds is described in U.S. Pat. No. 5,773,446, the entire disclosure of which is herein incorporated by reference.

The two enantiomers of iclaprim are known as 5-[(2R)-2-cyclopropyl-7,8-dimethoxy-2Hchromen-5-ylmethyl] pyrimidine-2,4-diamine (the “R-enantiomer”) and 5-[(2S)-2-cyclopropyl-7,8-dimethoxy-2Hchromen-5-ylmethyl] pyrimidine-2,4-diamine (the “S-enantiomer”), and have the structures shown below. Both R- and S-enantiomers have antibiotic activity.

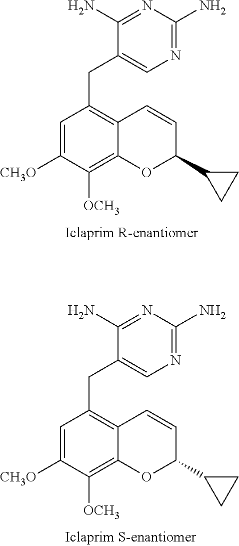

The iclaprim R- and S-enantiomers can be readily obtained or synthesized, for example by the method disclosed in C. Tahtaoui et al., Enantioselective Synthesis of Iclaprim Enantiomers; A Versatile Approach to 2-Substituted Chiral Chromenes, J. Org. Chem. (2010): 75, 3781-3785, the entire disclosure of which is herein incorporated by reference. The iclaprim racemate, the R-enantiomer or the S-enantiomer can be used in the present pharmaceutical formulations.

The R- and S-enantiomers of iclaprim both show antibiotic effects, but are not identical in terms of activity, pharmacokinetics or toxicity. For example, the R-enantiomer has a lower minimum inhibitory concentration (MIC) than the S-enantiomer, and is thus more potent against Gram-positive bacteria. The R-enantiomer also has more favorable pharmacokinetic parameters, and a reduced hERG channel activity (and thus lower expected cardiotoxicity), as compared to the S-enantiomer. The inventors have surprisingly found that the iclaprim R- and S-enantiomers also exhibit synergistic activity in combination with a sulfonamide antibiotic.

The iclaprim used in the pharmaceutical formulations of the invention can therefore comprise the racemate, the substantially isolated R-enantiomer, the substantially isolated S-enantiomer or a non-racemic mixture of the R-enantiomer and the S-enantiomer. In one embodiment, the iclaprim comprising the pharmaceutical formulations of the invention is substantially racemic. In another embodiment, the iclaprim comprising the pharmaceutical formulations of the invention is substantially the R-enantiomer. In another embodiment, the iclaprim comprising the pharmaceutical formulations of the invention is substantially the S-enantiomer.

Sulfonamide antibiotics, such as sulfamethoxazole, inhibit bacterial synthesis of dihydrofolic acid by competing with para-aminobenzoic acid (PABA), and are active against Gram-negative organisms. Iclaprim is a diaminopyrimidine derivative that is in the same pharmacological class as trimethoprim, and acts as a dihydrofolate reductase-inhibiting, extended-spectrum antibiotic active against Gram-positive organisms. Thus, sulfonamide antibiotics such as sulfamethoxazole and iclaprim block two consecutive steps in the biosynthesis of nucleic acids and proteins essential to bacterial growth.

The pharmaceutical formulations of the invention can comprise iclaprim or its enantiomers and a sulfonamide antibiotic in any amount suitable for treating a bacterial infection when administered to a subject. As used herein, a “subject” is any human or animal suspected of having, suffering from or at risk for acquiring a bacterial infection. In some embodiments, the sulfonamide antibiotic is present in the pharmaceutical formulations in a greater amount than the iclaprim. In other embodiments, the sulfonamide antibiotic and iclaprim are present in the pharmaceutical formulations in substantially equal amounts. In other embodiments, the iclaprim and sulfonamide antibiotic are present in the pharmaceutical formulation in a ratio of iclaprim to sulfonamide antibiotic of less than 1:5. For example, in some embodiments, the iclaprim and sulfonamide antibiotic are present in the pharmaceutical formulation in a ratio of iclaprim to sulfonamide antibiotic of about 1.1 to 4.5, for example about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4 and 1:4.5. In some embodiments, the iclaprim and sulfonamide antibiotic are present in the pharmaceutical formulation in a ratio of iclaprim to sulfonamide antibiotic of about 1:3, less than about 1:2 or less than about 1:1. In other embodiments, the iclaprim and sulfonamide antibiotic are present in the pharmaceutical formulation in a ratio of iclaprim to sulfonamide antibiotic of between about 1:0.5 to 1:3, 1:0.5 to 1:2 or 1:0.5 to 1:1. It is specifically contemplated that the iclaprim in the pharmaceutical formulations described in this paragraph can be an iclaprim enantiomer, for example substantially the R-enantiomer of iclaprim or substantially the S-enantiomer of iclaprim. In another embodiment, the pharmaceutical formulations described in this paragraph can be substantially the R-enantiomer.

The inventors have surprisingly found that the combination of iclaprim or its enantiomer, for example the R-enantiomer, with a sulfonamide antibiotic produces a synergistic antibiotic effect. See, for example, Example 2 below, which shows the antibacterial activity of different combinations of iclaprim and sulfamethoxazole tested against the analogous combinations of trimethoprim and sulfamethoxazole in an animal model of bacterial infection. Both combinations show a much greater reduction in bacterial load than with iclaprim alone, and the iclaprim/sulfamethoxazole combinations are more effective than the trimethoprim/sulfamethoxazole combinations when dosed at the same amounts. Iclaprim and trimethoprim are in the same class of antibiotics, and have similar targets and mechanisms of action. It is therefore unexpected that the iclaprim/sulfamethoxazole combinations show an increased antibacterial activity over the trimethoprim/sulfamethoxazole combinations when given at the same and lower doses, in particular when given at iclaprim to sulfonamide antibiotic ratios less than 1:5. Moreover, the synergistic effect of the iclaprim/sulfamethoxazole combinations is unexpectedly more pronounced at the lower iclaprim to sulfamethoxazole ratios.

The synergistic effect of the iclaprim/sulfonamide antibiotics is also shown in Example 4 below, in which iclaprim was tested in combination with a number of other antibiotics of different classes, to determine if the component antibiotics exhibited synergy, had no effect on each other, or inhibited each other's activity. As shown in Example 4, iclaprim in combination with sulfonamide antibiotics showed a synergistic effect, while iclaprim in combination with other antibiotics showed neither synergy nor inhibition of each other's activities.

The synergy of the iclaprim/sulfonamide combinations of the invention can be measured or described by any suitable method. For example, synergy can be expressed as the Fractional Inhibitory Concentrations (FIC) for each antibiotic in the combination can be calculated and used to determine the sum of FIC (ΣFIC) indicative of the synergistic potential of an iclaprim/sulfonamide antibiotic combination, for example as described in Veyssier P. (1999), Inhibiteurs de la dihydrofolate reductase, nitrohétérocycles (furanes) et 8-hydroxyquinoleines, pp. 995-1027, in A. Bryskier (ed.), Antibiotiques agents antibatériens et antifongiques, 1st Ed., Ellipses Édition Marketing SA, Paris, the entire disclosure of which is herein incorporated by reference. Synergy of the iclaprim/sulfonamide combinations of the invention can therefore be defined as where the ΣFIC of the combination is <0.5; indifference (no synergy nor antagonism) can be defined as where the ΣFIC of the combination is ≥0.5 but ≤4; and antagonism can be defined as where the ΣFIC of the combination is >4. Exemplary calculations of ΣFIC by this method are shown in Example 4 below.

The pharmaceutical formulations of the invention can be formulated into any suitable dosage form for administration to a subject. For example, the present pharmaceutical formulations can be formulated into one or more dosage forms for oral administration, for example as a tablet or caplet, for intravascular or intramuscular administration, for rectal administration (for example as a suppository), for sublingual administration or for inhalation. In one embodiment, the pharmaceutical formulations are formulated for intravenous administration.

One skilled in the art would understand how to manufacture the present pharmaceutical formulations, and how to formulate them into one or more dosage forms. For example, the present pharmaceutical formulations can be manufactured by mixing powdered iclaprim or its enantiomers, for example the R-enantiomer, and sulfonamide antibiotic in the desired amounts, either neat or with any suitable pharmaceutical excipients. The amount and nature of the pharmaceutical excipients to be mixed with the iclaprim or its enantiomers, for example the R-enantiomer, and sulfonamide antibiotic can be readily determined by one of ordinary skill in the art, for example by considering the solubility and other known physical characteristics of the iclaprim or its enantiomers, for example the R-enantiomer, and sulfonamide antibiotic, and the chosen route of administration. Thus, in some embodiments, the present disclosure provides methods of manufacturing a pharmaceutical formulation, or one or more dosage forms thereof, for treating a bacterial infection in a subject, comprising combining iclaprim or its enantiomers, for example the R-enantiomer, and a sulfonamide antibiotic. In still other embodiments, the present disclosure provides the use of iclaprim or its enantiomers, for example the R-enantiomer, and a sulfonamide antibiotic to manufacture a medicament, or one or more dosage forms thereof, for the treatment of a bacterial infection in a subject. As used herein, “medicament” is meant to be equivalent to “pharmaceutical formulation,” and both terms are used interchangeably.

Thus, in some embodiments, the pharmaceutical excipients used to formulate a pharmaceutical formulation or dosage form of the invention can be solid and/or liquid. Suitable liquid excipients are well known and may be readily selected by one of skill in the art. Such excipients can include, for example, liquid carriers such as water, DMSO, saline, buffered saline, lactated Ringer's solution, Ringer's acetate solution, hydroxypropylcyclodextrin solutions or ethanolic solutions. Other suitable pharmaceutical excipients which can be used to make liquid pharmaceutical formulations of the invention include metal chelators, osmo-regulators, pH adjustors, preservatives, solubilizers, sorbents, stabilizers, sweeteners, surfactants, suspending agents, syrups, thickening agents and/or viscosity regulators. The liquid pharmaceutical excipients can be sterile solutions, for example when used for preparing pharmaceutical formulations for parenteral (e.g., intravenous) administration.

In some embodiments, pharmaceutical formulations of the invention can be formulated with liquid pharmaceutical excipients to form solutions, suspensions, emulsions, syrups or elixirs. In one embodiment, the iclaprim or its enantiomers, for example the R-enantiomer, and sulfonamide antibiotic is dissolved in a liquid carrier to form a solution. In another embodiment, the iclaprim or its enantiomers, for example the R-enantiomer, and sulfonamide antibiotic is suspended in a liquid carrier to form a suspension. In another embodiment, the iclaprim or its enantiomers, for example the R-enantiomer, is dissolved in the liquid carrier and the sulfonamide antibiotic is suspended in the liquid carrier.

Suitable solid excipients for formulating the pharmaceutical formulations of the invention are also well known to those of ordinary skill in the art. A given solid excipient can perform a variety of functions; i.e., one substance can perform the functions of two or more of the excipients described below. For example, a solid excipient can act both as a filler and a compression aid. Examples of solid excipients which can comprise a pharmaceutical formulation of the invention include: adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants and/or bulking agents.

In one embodiment, the iclaprim or its enantiomers, for example the R-enantiomer, and sulfonamide antibiotic can be formulated with one or more solid pharmaceutical excipients and compacted into a unit dose form; i.e., a tablet or caplet. In another embodiment, the iclaprim or its enantiomers, for example the R-enantiomer, and sulfonamide antibiotic can be formulated neat or with one or more solid pharmaceutical excipients as powder or granules and added to unit dose form; i.e., a capsule. In one embodiment, the iclaprim or its enantiomers, for example the R-enantiomer, and sulfonamide antibiotic can be formulated neat or with one or more solid pharmaceutical excipients as a powder.

For a discussion of the properties of solid and liquid pharmaceutical excipients which are suitable for use in the present pharmaceutical formulations, see, e.g., the excipients described in the Rowe et al., eds., Handbook of Pharmaceutical Excipients, 7th Edition, London: Pharmaceutical Press, 2012, which is incorporated herein by reference.

The solid or liquid pharmaceutical formulations of the invention can be formulated or divided into one or more dosage forms for subsequent administration to a subject. For example, the one or more dosage forms can be packaged compositions; e.g., packeted powders (sachets), vials, ampoules, prefilled syringes or bags. Other suitable dosage forms include pre-formed dosage forms such as tablets, caplets, capsules or suppositories. In one embodiment, the one or more dosage forms is a tablet. In another embodiment, the one or more dosage forms is a tablet comprising a pharmaceutical formulation of the invention and further comprising a surfactant, a lubricant, a disintegrant, a diluent or a binder. For example, the tablet can comprise a pharmaceutical formulation of the invention and docusate sodium as a surfactant, sodium benzoate as a lubricant, sodium starch glycolate as a disintegrant, magnesium stearate as a diluent and pregelatinized starch as a binder.

The pharmaceutical formulations of the invention can also be provided in dry or lyophilized forms, or as a liquid concentrate, for subsequent reconstitution or dilution into a dosage form by the addition of a suitable liquid pharmaceutical excipient. For example, a powdered, lyophilized or concentrated liquid pharmaceutical formulation of the invention can be provided in a container, to which a sterile liquid pharmaceutical excipient is added prior to (for example, immediately prior to) parenteral, e.g., intravenous, administration to a subject.

In one embodiment, the pharmaceutical compositions of the invention can be utilized as inhalants. For this route of administration, the pharmaceutical compositions can be prepared as fluid unit doses comprising a vehicle suitable for delivery by an atomizing spray pump or by dry powder for insufflation. In another embodiment, the pharmaceutical compositions of the invention can be delivered as aerosols; i.e., orally or intranasally. For this route of administration, the pharmaceutical compositions can be formulated for use in a pressurized aerosol container together with a gaseous or liquefied propellant; e.g., dichlorodifluoromethane, carbon dioxide, nitrogen, propane, and the like, for example by delivery as a metered dose in one or more actuations from a suitable delivery device.

The pharmaceutical formulations or dosage forms of the invention can be administered to a subject by any suitable route, taking into consideration factors such as the age, weight and the overall condition of the subject, the type of bacterial infection to be treated, and the like. For example, the pharmaceutical formulations or dosage forms of the invention can be delivered orally, by injection, transdermally, intravascularly (e.g., intra-arterially or intravenously), subcutaneously, intramuscularly, intra-articularly, sublingually, topically, intranasally, by inhalation, rectally, and vaginally, among others.

The pharmaceutical formulations or dosage forms of the invention can contain any amount of iclaprim or its enantiomers, for example the R-enantiomer, and sulfonamide antibiotic suitable for treating a bacterial infection. In one embodiment, the pharmaceutical formulations or dosage forms of the invention can comprise about 100 to 1600 mg of sulfonamide antibiotic, for example about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500 or 1600 mg of sulfonamide antibiotic. In one embodiment, the pharmaceutical formulations or dosage forms of the invention can comprise about 20 to 320 mg of iclaprim or its enantiomers, for example the R-enantiomer, for example about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 220, 300 or 320 mg. In other embodiments, the amount of iclaprim or its enantiomers, for example the R-enantiomer, comprising the pharmaceutical formulations or dosage forms of the invention is calculated as about ½ to ¼, for example about ½, ⅓ or ¼ the amount of sulfonamide antibiotic present.

In one embodiment, the pharmaceutical formulations or dosage forms of the invention comprise about 160 mg of iclaprim or its enantiomers, for example the R-enantiomer, and about 160 mg of sulfonamide antibiotic. In another embodiment, the pharmaceutical formulations or dosage forms of the invention comprise about 160 mg of iclaprim or its enantiomers, for example the R-enantiomer, and about 320 mg of sulfonamide antibiotic. In another embodiment, the pharmaceutical formulations or dosage forms of the invention comprise about 160 mg of iclaprim or its enantiomers, for example the R-enantiomer, and about 480 mg of sulfonamide antibiotic.

The pharmaceutical formulations and dosage forms of the invention can be administered to a subject to treat a bacterial or fungal infection caused by any organism susceptible to iclaprim and/or a sulfonamide antibiotic, for example sulfamethoxazole. One of ordinary skill in the art is aware of or can readily determine which bacteria or fungi are susceptible to iclaprim and/or a sulfonamide antibiotic. For example, the following bacteria are susceptible to pharmaceutical formulations and dosage forms of the invention: aerobic gram-positive microorganisms such as Streptococcus pneumoniae (including penicillin-resistant Streptococcus pneumoniae) and Staphylococcus aureus (including methicillin-resistant Staphylococcus aureus; aerobic gram-negative microorganisms such as Escherichia coli (including susceptible enterotoxigenic strains implicated in traveler's diarrhea), Klebsiella species, Enterobacter species, Haemophilus influenzae, Morganella morganii, Proteus mirabilis, Proteus vulgaris, Shigella flexneri and Shigella sonnei. For example, fungi such as Pneumocystis jiroveci are also susceptible to pharmaceutical formulations and dosage forms of the invention.

Any suitable testing methods known in the art, such as dilution techniques and diffusion techniques, can be used to determine whether any bacteria is susceptible to the pharmaceutical formulations and dosage forms of the invention. Dilution techniques are quantitative methods used to determine antimicrobial minimum inhibitory concentrations (MICs) of antibacterial compounds diluted into solutions such as broth or agar. MICs determined in this manner can provide an indication of the susceptibility of bacteria to the antimicrobial compounds being tested. A suitable dilution technique for determining MICs is disclosed in Clinical and Laboratory Standards Institute, Performance Standards for Antimicrobial Disk Susceptibility Tests; Approved Standard—11th ed. CLSI document M02-A11, CLSI, Wayne, Pa. (2012), the entire disclosure of which is herein incorporated by reference.

Diffusion techniques are quantitative methods that measure zones of bactericidal or bacteriostatic activity, for example in a diameter around a paper disk soaked in a solution containing the antimicrobial compounds being tested, which disk has been contacted with a bacterial lawn. Such techniques can provide reproducible estimates of the susceptibility of a given bacteria to antimicrobial compounds, measured as a function of the growth inhibition or bacterial death- or growth inhibition-zone size. A suitable diffusion technique is disclosed in Clinical and Laboratory Standards Institute (CLSI), Performance Standards for Antimicrobial Susceptibility Testing; Twenty-third Informational Supplement (CLSI document M100-S23), Clinical and Laboratory Standards Institute, Wayne, Pa. (2013), the entire disclosure of which is herein incorporated by reference.

Thus, the invention provides a method of treating a bacterial infection in a subject, comprising administering to a subject a therapeutically effective amount of a pharmaceutical formulation of the invention, or one or more dosage forms thereof. In one embodiment, the treatment methods of the invention include the step of determining whether the bacteria causing the infection in the subject is susceptible to the iclaprim or its enantiomers, for example the R-enantiomer, and/or sulfonamide antibiotic comprising the pharmaceutical formulations or dosage forms of the invention.

The bacterial infections that can be treated with the methods of the invention include any infection caused by a bacteria which is susceptible to iclaprim or its enantiomers, for example the R-enantiomer, and/or a sulfonamide antibiotic, for example sulfamethoxazole. In some embodiments, the bacterial infections that can be treated with the methods of the invention include urinary tract infections, otitis media, bronchitis, Shigellosis, pneumonia, traveler's diarrhea or a skin and structure infection. In certain embodiments, the pneumonia may comprise hospital-acquired bacterial pneumonia or ventilator-associated bacterial pneumonia.

As used herein, a “therapeutically effective amount” of a pharmaceutical formulation of the invention, or one or more dosage forms thereof, is any amount which treats the bacterial infection. As used herein, to “treat” a bacterial infection means that bactericidal or bacteriostatic activity is observed, and/or that one or more symptoms of the bacterial infection (e.g., redness, swelling, increased temperature of the infected area, presence of pus, fever, aches, chills, and the like) are reduced, ameliorated or delayed.

The ordinarily skilled physician can readily determine the therapeutically effective amounts of the a pharmaceutical formulation or dosage form of the invention for administration to a subject according to the present methods, for example by taking into account factors such as the specific bacterial infection to be treated, and the size, age, weight, gender, disease penetration, route of administration, previous treatments and response pattern of the subject.

In some embodiments, therapeutically effective amounts of the a pharmaceutical formulation or dosage form of the invention include about 1.6 to 12.8 mg of sulfamethoxazole per kg of body weight and about 0.4 to 3.2 mg iclaprim or its enantiomers, for example the R-enantiomer, per kg of body weight in a 24 hour period.

Suitable dosages and ratios of the combination treatment of iclaprim and sulfonamide may be determined based on the relative pharmacokinetics and plasma half-life (t½) of iclaprim and the selected sulfonamide. The present inventors have studied the pharmacokinetics of the combination treatment sulfamethoxazole and trimethoprim. Trimethoprim is a weak base with a pKa of 7.3, a t½ of about 10.1 hours and is widely distributed in the body after oral administration. Similarly, sulfamethoxazole has a pKa of 6.0, a t½ of about 11.4 hours and has lower levels of distribution in tissue fluids than trimethoprim. Due to the greater volume distribution of trimethoprim compared to sulfamethoxazole, a trimethoprim: sulfamethoxazole dosage ratio of 1:5 may result in a plasma ratio of 1:20. While iclaprim is structurally related to trimethroprim, it has a t½ of about 2.9 hours. As a consequence of the shorter half-life of iclaprim relative to sulfamethoxazole, a dosage ratio of iclaprim:sulfamethoxazole of 1:5 may result in a plasma ratio much greater than 1:20.

Accordingly, if compensation for differences in volume distribution is desired, the dosage ratio of iclaprim:sulfonamide may be adjusted based on the pharmacokinetic profile of the selected sulfonamide. Alternatively or additionally, a sulfonamide having a suitable half life may be selected for combination treatments with iclaprim. Suitably, the half-life (t½) of the sulfonamide administered in combination with iclaprim to a human patient may be 11 hours or less, or more preferably 7 hours or less, or even more preferably 4 hours or less.

A therapeutically effective amount of the pharmaceutical formulations or dosage forms of the invention can be administered to a subject on regular schedule; i.e., a daily, weekly or monthly at regular intervals, or on an irregular schedule with varying administration over days, weeks, or months. Alternatively, the therapeutically effective amount of the present pharmaceutical formulations or dosage forms administered can vary between administrations. For example in one embodiment, the amount for the first administration is higher than the amount for one or more of the subsequent administrations. In another embodiment, the amount for the first administration is lower than the amount for one or more of the subsequent administrations.

A therapeutically effective amount of the pharmaceutical formulations or dosage forms of the invention can be administered over various time periods, for example about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a course of therapy can be determined according to the judgment of the ordinarily-skilled physician. The therapeutically effective amounts described herein can refer to a single administration of the pharmaceutical formulations or dosage forms of the invention, or can refer to the total amounts administered for a given time period.

In some embodiments, effective amounts of the pharmaceutical formulations or dosage forms of the invention are administered to a subject, for example as equally divided doses or as unequally divided doses, about every 24 hours for 5 days; every 12 hours for 3 days; every 12 hours for 5 days; every 6 hours for 10 to 21 days; every 12 hours for 14 days, every 12 hours for 10 days; 3 or 4 equally divided doses every 6 to 8 hours for up to 14 days; 2 or 4 equally divided doses every 6, 8 or 12 hours for up to 14 days. In another embodiment, effective amounts of the pharmaceutical formulations or dosage forms of the invention are administered to a subject, for example as equally divided doses or as unequally divided doses, for about 5 to 14 days, from one to three times a day.

Also provided herein are kits comprising one or more dosage forms comprising pharmaceutical formulations of the invention. The kits can optionally comprise instructions to direct a health care professional or a subject to prepare, store and/or administer the one or more dosage forms.

Suitably, the kit contains packaging or a container with the one or more dosage forms formulated for the desired route of administration. Other suitable components comprising kits of the invention will be readily apparent to one of skill in the art, taking into consideration the desired indication, type of dosage form and the desired delivery route. A number of packages or containers are known in the art for dispensing the one or more dosage forms. In one embodiment, the package comprises indicators to assist in monitoring the delivery schedule for the one or more dosage forms. In another embodiment, the package comprises a blister package, dial dispenser package, bottle, vial, ampoule or flexible bag.

The packaging comprising a kit of the invention can itself be engineered to perform or assist in the administration of the one or more dosage forms, and can comprise for example a catheter, syringe, pipette, flexible IV bag optionally with tubing, metered dosing device for inhalation or insufflation or other apparatus from which the one or more dosage forms can be applied to or into the subject, or to or into an affected area of the subject.

In some embodiments, the one or more dosage forms comprising kits of the invention are provided in dried, lyophilized or concentrated forms. In such embodiments, the kits of the invention can further comprise reagents or components for reconstitution or dilution of the one or more dosage forms. In other embodiments, the kits of the invention can comprise a means for containing the one or more dosage forms in close confinement for, e.g., commercial sale, such as injection or blow-molded plastic containers into which the one or more dosage forms are retained.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are illustrative only, since alternative methods can be utilized to obtain similar results.

### Example 1—Reduction of Bacterial Load with Iclaprim Racemate

The ability of the pharmaceutical formulations of the invention to treat bacterial infections was further demonstrated using a mouse subcutaneous abscess model in which the abscesses were induced with Staphylococcus aureus AH 1246. Racemic iclaprim and sulfamethoxazole formulations at 8 and 15 mg/kg (with respect to the iclaprim), at iclaprim to sulfamethoxazole ratios of 1:1, 1:3 and 1:5, were prepared and tested against the analogous combinations of trimethoprim and sulfamethoxazole. Iclaprim alone at 40 mg/kg was used as a control.

Bacterial Growth Media:

Trypticase Soy Agar (TSA) plates—BBL, Franklin Lakes, N.J., USA; Brain Heart Infusion (BHI) Broth—BBL, Franklin Lakes, N.J., USA.

Cytodex® Beads:

Sigma Aldrich, St. Louis, Mo., USA.

MIC determinations for the strains employed in these studies were performed using standard CLSI microdilution techniques.

Bacterial Strains:

Wild type S. aureus ATCC 25923 and its TK-deficient mutant AH 1246 were supplied by Arpida AG, Reinach, Switzerland. TK mutants were derived as described by Haldimann A, et al., Effect of Thymidine on the Activity of Diaminopyrimidine Antibacterial Agents: Generation and Characterization of Thymidine Kinase-Deficient Staphylococcus aureus Mutants. 46th Interscience Conference on Antimicrobial Agents and Chemotherapy (2006), Abstract C1-940, the entire disclosure of which is herein incorporated by reference.

Methods and Experimental Design

Bacteria:

S. aureus strains were grown on TSA plates at 37° C. in 5% CO2. The bacteria concentration was adjusted by re-suspending a portion of the overnight growth of the plate in saline and adjusting a 1:10 dilution of the suspension to achieve an OD625 of 0.1. The adjusted suspension was diluted 1:2 in prepared Cytodex beads (1 gram/50 mL PBS) to a final concentration of 5.0×105 CFU/mL. Bacterial enumeration was performed to determine actual concentration of the bacterial inoculum.

Animals:

CD-1 female mice (weighing 18 to 22 grams) from Charles River Laboratories (Wilmington, Mass.) were acclimated for 5 days prior to start of study. All studies were performed under approved IACUC protocols and conform to OLAW standards. Animals had free access to food and water throughout the study. Animals were provided enrichment and housed 5 per cage.

Infection Studies:

Mice were injected SC with 0.2 mL of the bacterial-Cytodex inoculum. At 8, 24, 32, 48 and 56 hours post infection, the mice were treated with a single dose of the iclaprim/sulfamethoxazole combinations or the iclaprim control. The mice were then euthanized and the abscesses aseptically removed, homogenized, serially diluted and plated for bacterial enumeration. Mean values and standard deviations for the change in average log 10 CFU/gr between treatment and control mice were calculated, and the results are shown in Table 2.

As can be seen from Table 2, both the iclaprim/sulfamethoxazole and the trimethoprim/sulfamethaxazole formulations reduced bacterial load of thymidine kinase deficient mutant S. aureus AH1246 in the mice. However, the iclaprim/sulfamethoxazole formulations showed a greater antibacterial activity than the analogous trimethoprim/sulfamethaxazole formulations when given at the same doses, in particular at the 1:1 and 1:3 iclaprim to sulfamethoxazole ratios. Such results were unexpected as iclaprim and trimethoprim are in the same class of antibiotics and have similar targets and mechanisms of action.

TABLE 2

| | | Change in average | | |
|---|---|---|---|---|
| | | log10 CFU/g of Abscess | | |
| | Conc. of | from Controls | | |
| | Icla/trimeth | 1:5 | 1:3 | 1:1 |
| | (mg/Kg) | ratio | ratio | ratio |
| Icla:Sulfa | 8 | −1.75 | −2.15 | −2.20 |
| | 15 | −2.79 | −3.20 | −2.64 |
| Trimeth:Sulfa | 8 | −1.61 | −1.90 | −1.30 |
| | 15 | −2.26 | −2.33 | −1.85 |

| | | Change in average |
|---|---|---|
| | | log10 CFU/gr of Abscess |
| | | from Controls |
| Iclaprim | 40 (alone) | −0.79 |

PO dosing @ 8, 24, 32, 48, 56 hours post infection

### Example 2—Reduction of Bacterial Load with Iclaprim Enantiomers

The ability of the pharmaceutical formulations of the invention to treat bacterial infections can be demonstrated using the mouse subcutaneous abscess model described above in Example 1, but testing iclaprim R-enantiomer and S-enantiomer and sulfamethoxazole formulations at 8 and 15 mg/kg (with respect to the iclaprim) in addition to racemic iclaprim and sulfamethoxazole formulations at 8 and 15 mg/kg, at iclaprim (enantiomer or racemate) to sulfamethoxazole ratios of 1:1, 1:3 and 1:5. The iclaprim enantiomer and racemate formulations may be tested against the analogous combinations of trimethoprim and sulfamethoxazole, and iclaprim racemate alone at 40 mg/kg may be used as a control.

### Example 3—In Vitro and In Vivo Activity Anti-Pneumocystis Activity of Iclaprim/Sulfamethoxazole Combination

The activity of an iclaprim/sulfamethoxazole (SMX) combination against Pneumocystis was tested in vitro using an efficient axenic culture system and in vivo using P. jirovecii-endotracheally inoculated corticosteroid-treated rats, the most reproducible Pneumocystis pneumonia (PcP) model available to date. Animals were orally administered with iclaprim (5, 25 or 50 mg/kg/d), iclaprim/SMX (5/25, 25/125 or 50/250 mg/kg/d), trimethoprim (TMP) (50 mg/kg/d) or TMP/SMX (50/250 mg/kg/d) once a day for 10 consecutive days.

Materials and Methods

Drugs.

Iclaprim was synthesized at Arpida Ltd. (Münchenstein, Switzerland). TMP and SMX were obtained from Sigma, Spain. Iclaprim, TMP and SMX were dissolved in 100% dimethyl sulfoxide (DMSO, Sigma) to produce stock solutions of 100, 30 and 150 mg/ml, respectively. TMP and SMX solutions were mixed appropriately to obtain a final 1:5-combination. For evaluating the in vitro anti-Pneumocystis activity, the drug stock solutions were diluted in Dulbecco's Modified Eagle's Medium (DMEM, Bio-Whittaker) supplemented with 10% heat-inactivated fetal calf serum (FCS, GIBCO-BRL) to produce the required drug concentrations. To evaluate the in vivo anti-Pneumocystis activity, additional iclaprim-, TMP- or SMX-stock solutions were made and used: Drug solutions of 100 mg/ml were used for iclaprim and TMP, and a solution of 500 mg/ml was used for SMX. Then the drug stock solutions were diluted in sterile water before gavages. Compound solutions were prepared just before use.

Source of Pneumocystis jirovecii.

Corticosteroid-treated conventional laboratory rats were used as an animal model to obtain P. jirovecii organisms. Ten-week-old female Wistar rats (Harlan, France) were immunosuppressed for 3 weeks with dexamethasone (Fortecortin®, Merck) administered in the drinking water (2 mg/liter). Rats were then inoculated with 20×10⁶ of cryopreserved parasites using a non-surgical endotracheal method. Dexamethasone treatment was maintained until the end of the experiment. Six to eight weeks' post-inoculation (p.i.) rats were highly infected, without secondary fungal or bacterial infection. Animals were allowed sterile standard food (UAR, France) and water ad libitum. The research complied with national legislation and with company policy on the Care and Use of Animals and with the related code of practice.

Extraction, Purification and Quantitation of P. jirovecii.

Six to eight weeks following inoculation, rats were sacrificed and parasite extraction was performed. Briefly, parasites were extracted in Dulbecco's Modified Eagle's Medium (DMEM; Bio-Whittaker) by agitation of lung pieces with a magnetic stirrer. The resulting homogenate was poured successively through gauze, 250 and 63 micron stainless steels filters. After centrifugation, the pellet was resuspended in a hemolytic buffered solution. P. jirovecii organisms were collected by centrifugation and then purified on a polysucrose gradient (Histopaque-1077, Sigma Chemical Co.). Blood and Sabouraud dextrose agar (Difco) media were inoculated with purified parasites to check for the presence of eventual contaminating pathogens. P. jirovecii was quantitated on air dried smears stained with RAL-555 (Reactifs RAL, France), a rapid panoptic methanol-Giemsa stain, which stains trophozoites, precysts and cysts of P. jrovecii.

In Vitro Susceptibility Study.

In vitro pharmacodynamic properties were determined using the Hill equation (Eₘₐₓ sigmoid model; see below). This approach offers at least 3 parameters which can be used to describe the in vitro activity of new therapeutic compounds: the maximum effect (Eₘₐₓ) as a measure for efficacy, the 50% effective concentration (EC₅₀) as a parameter of intrinsic activity, and the slope (S) of the concentration-effect relationship.

In vitro susceptibility studies were performed using the broth microdilution technique. Final drug concentrations ranged from 100 to 5 μg/ml for iclaprim; 5/25 to 100/500 μg/ml for the combination iclaprim/SMX, and 1/5 to 150/750 μg/ml for the combination TMP/SMX. All the experiments were carried out in 24-well plates with a final volume of 2 ml of DMEM supplemented with 10% FCS containing a final inoculum of 0.5×10⁶ organisms per ml. Plates were then incubated for 4 days in an atmosphere of 5% C0² at 37° C. One free-drug control was included in each assay. Parasite quantitation was performed on homogenate smears as described above. All susceptibility assays were set up in triplicate.

Analysis of results. The in vitro activity of tested compounds against P. jirovecii was expressed as percentage of inhibition defined as: the total parasite number found in drug-treated wells in comparison with parasite counts in control wells without drug. Once all the differences between drug-treated and untreated wells were calculated, the concentration-effect relationship was established by using the Hill equation:

$${E}_{R} = \frac{{E}_{R , ma x} \cdot {C}^{S}}{[ {( {EC}_{50} )}^{S} + {C}^{S} ]}$$

where E_{R} is the effect of each drug concentration (C) on the percentage of inhibition estimated from experimental results; S is a parameter reflecting the steepness of the concentration-effect relationship curve; EC₅₀ is the concentration of the compound at which 50% of the maximum effect (E_{R,max}) is obtained. The parameters of this pharmacodynamic model were calculated by nonlinear least-squares regression techniques using a commercial software (Sigma Plot).

In Vivo Susceptibility Study.

An in vivo experiment with corticosteroid-treated Wistar rats endotracheally inoculated with P. jirovecii was performed in order to explore whether in vitro results reflected in vivo efficacy. Animals were divided into groups of 3, and then orally dosed with iclaprim at 5, 25 or 50 mg/kg; TMP at 50 mg/kg, TMP/SMX at 50/250 mg/kg (diluted in DMSO) or using Bactrim® oral solution (Roche, France); and the combination iclaprim/SMX at 5/25, 25/125 or 50/250 mg/kg. The drugs were given once a day for 10 consecutive days. The final concentration of DMSO in diluted drug solutions was between 1.5 and 15%. Control animals were dosed with sterile water with 15% of DMSO. At the end of the experiment, therapeutic efficacy was assessed by counting P. jirovecii in lung homogenates and comparing the counts with those of the untreated controls. Twenty-four hours after the end of the treatment, animals were sacrificed and the lung homogenized in a Stomacher-400 blender as previously described (European Concerted Action on Pneumocystis Research. Parasitology Today 12: 245-9, 1996, the entire disclosure of which is herein incorporated by reference). Parasite quantitation was performed on air-dried smears stained with toluidine blue 0 (cystic forms) or RAL-555 stains (vegetative, precystic and cystic forms).

Results

In Vitro Susceptibility Study.

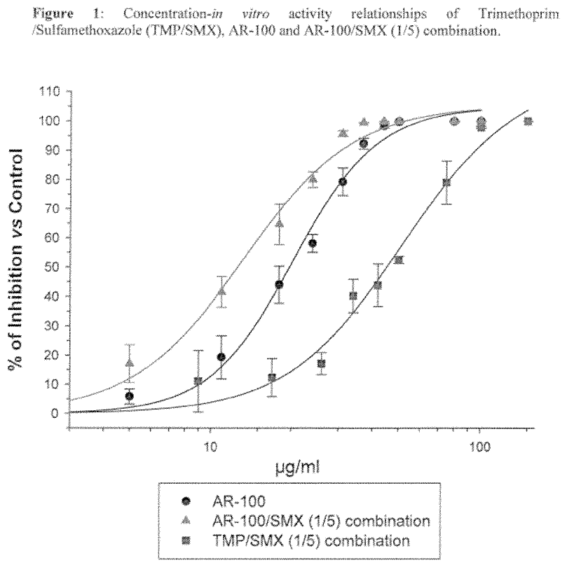

FIG. 1 shows concentration-response curves obtained after 4 days of incubation of P. jirovecii with iclaprim or the combinations iclaprim/SMX and TMP/SMX. The reduction in the number of microorganisms was gradual and concentration dependent. TMP/SMX demonstrated the lowest intrinsic activity with an EC₅₀ of 51.4/257 μg/ml. Iclaprim alone had a high in vitro anti-Pneumocystis activity, with an EC₅₀ value of 20.3 μg/ml. The iclaprim/SMX combination (proportion 1:5) showed a significant synergistic activity, with an EC₅₀ value of 13.2/66 μg/ml. In terms of efficacy, the combination iclaprim/SMX at a concentration of 37/185 μg/ml (See Table 1 below). However, higher concentrations of TMP/SMX (150/750 μg/ml) were needed for reaching a ˜99% inhibition.

In Vivo Susceptibility Study.

Untreated animals were highly infected at the end of the treatment period. The number of total P. jirovecii organisms per lung was 2.2±0.3 109 (see Table 2 below). The combination TMP/SMX diluted in DMSO showed similar anti-Pneumocystis activity to Bactrim® (86.6±7.1% versus 96.8±2.6%). Iclaprim and TMP showed a similar activity at the concentration of 50 mg/kg/day. The iclaprim/SMX combination was more potent (98.5±0.9% of inhibition for 25/125 mg/kg/day) than TMP/SMX (86.6±7.1% of inhibition for 50/250 mg/kg/day). All the tested drugs affected trophozoites as well as cystic forms of P. jirovecii (See Table 2).

Discussion

The iclaprim/SMX combination (proportion 1:5) showed a significant synergistic activity, with an EC₅₀ value of 13.2/66 μg/ml. The TMP/SMX combination was the least potent compound tested (EC₅₀ of 51/255 μg/ml). In vivo, though iclaprim and TMP showed a similar activity, the iclaprim/SMX combination was more potent (98.5±0.9% of inhibition for 25/125 mg/kg/d) than TMP/SMX (86.6+7.1% of inhibition for 50/250 mg/kg/d). Thus, the iclaprim/SMX combination showed considerably more anti-Pneumocystis activity than TMP/SMX, indicating that the synergistic iclaprim/SMX combination could constitute an advantageous therapeutic alternative to the use of TMP/SMX for treating severe forms of PcP in humans. Moreover, careful differential parasite counts performed on RAL-555-stained smears has shown that iclaprim alone or combined with SMX inhibited the growth of both Pneumocystis cysts and vegetative forms. This was an important difference with other anti-Pneumocystis drugs like echinocandin-derived compounds, which are inhibitors of the β-1,3 glucan synthesis and which selectively eliminate cysts in infected rats submitted to therapeutic doses.

TABLE 1
Concentration-in vitro activity relationships of Trimethoprim/Sulfamethoxazole(TMP/SMX; 1:5), Iclaprim and Iclaprim/SMX

| | Total | | % | | | | | Iclaprim/ | Total | | |
|---|---|---|---|---|---|---|---|---|---|---|---|
| TMP/SMX | parasites | | inhibition | Iclaprim | Total parasites | | % inhibition | SMX | parasites | | % inhibition |
| (μg/ml) | (× 10⁶) | Log | vs control | (μg/ml) | (× 10⁶) | Log | vs control | (μg/ml) | (× 10⁶) | Log | vs control |
| 0 | 3.4 ± 0.3 | 6.5 | 0.0 ± 8.5 | 0 | 1.6 ± 0.1 | 6.2 | 0.0 ± 8.3 | 0 | 1.6 ± 0.1 | 6.2 | 0.0 ± 5.8 |
| 1/5 | 3.4 ± 0.3 | 6.5 | −2.3 ± 7.9 | 5 | 1.5 ± 0.04 | 6.2 | 5.8 ± 2.6 | 5/25 | 1.3 ± 0.1 | 6.1 | 17.1 ± 6.5 |
| 9/46 | 3.0 ± 0.4 | 6.5 | 11.1 ± 10.6 | 11 | 1.3 ± 0.1 | 6.1 | 19.3 ± 7.4 | 11/55 | 0.9 ± 0.08 | 5.9 | 41.6 ± 5.2 |
| 17/87 | 2.9 ± 0.2 | 6.5 | 12.4 ± 6.5 | 18 | 0.9 ± 0.1 | 5.9 | 44.0 ± 6.3 | 18/90 | 0.6 ± 0.1 | 5.7 | 64.6 ± 7.0 |
| 26/128 | 2.8 ± 0.1 | 6.4 | 17.2 ± 3.8 | 24 | 0.7 ± 0.05 | 5.8 | 58.1 ± 3.1 | 24/120 | 0.3 ± 0.04 | 5.5 | 80.0 ± 2.7 |
| 34/168 | 2.0 ± 0.2 | 6.3 | 40.2 ± 5.7 | 31 | 0.3 ± 0.08 | 5.5 | 79.3 ± 4.8 | 31/155 | 0.07 ± 0.02 | 4.8 | 95.7 ± 1.0 |
| 42/209 | 1.9 ± 0.3 | 6.3 | 43.8 ± 7.3 | 37 | 0.1 ± 0.03 | 5.1 | 92.3 ± 1.9 | 37/185 | 0.01 ± 0.001 | 3.9 | 99.4 ± 0.04 |
| 50/250 | 1.6 ± 0.04 | 6.2 | 52.4 ± 1.2 | 44 | 0.03 ± 0.01 | 4.4 | 98.3 ± 0.72 | 44/220 | 0.003 ± 0.001 | 3.5 | 99.8 ± 0.06 |
| 75/375 | 0.7 ± 0.3 | 5.8 | 79.0 ± 7.4 | 50 | 0.01 ± 0.001 | 3.7 | 99.7 ± 0.04 | 50/250 | 0.002 ± 0.001 | 3.1 | 99.9 ± 0.08 |
| 100/500 | 0.07 ± 0.01 | 4.9 | 97.8 ± 0.3 | 80 | 0.003 ± 0.002 | 3.4 | 99.8 ± 0.12 | 80/400 | 0.002 ± 0.001 | 3.2 | 99.9 ± 0.08 |
| 150/750 | 0.004 ± 0.001 | 3.6 | 99.9 ± 0.03 | 100 | 0.002 ± 0.001 | 3.3 | 99.9 ± 0.07 | 100/500 | 0.001 ± 0.001 | 3.0 | 99.9 ± 0.05 |

TABLE 2
In vivo of Iclaprim/SMX or TMP/SMX combinations on rat delivered Pneumocystis jirovecii

| | | Total | % of | | Total | % of | Mean |
|---|---|---|---|---|---|---|---|
| | Rat | cysts | inhibition | Mean % + | parasites | inhibition | % + |
| Groups | Number | (TBO) | vs control | SD | (Giemsa) | vs Control | SD |
| 1-Control | 1.1 | 6.96.10⁺⁷ | / | / | 1.82.10⁺⁹ | / | / |
| | 1.2 | 1.47.10⁺⁸ | / | / | 2.10.10⁺⁹ | / | / |
| | 1.3 | 1.45.10⁺⁸ | / | / | 2.63.10⁺⁹ | / | / |
| 2- | 2.1 | 2.58.10⁺⁷ | 78.6 | 82.5 ± 5.3 | 1.38.10⁺⁸ | 93.7 | 86.6 ± 7.1 |
| TMP/SMX | | | | | | | |
| 50-250 mg/kg/d | 2.2 | 1.38.10⁺⁷ | 88.5 | | 2.95.10⁺⁸ | 86.5 | |
| | 2.3 | 2.37.10⁺⁷ | 80.3 | | 4.46.10⁺⁸ | 79.6 | |
| 3-TMP | 3.1 | 7.16.10⁺⁷ | 40.6 | 64.2 ± 30.7 | 1.06.10⁺⁹ | 51.3 | 78.7 ± 22.6 |
| 50 mg/kg/d | 3.2 | 9.36.10⁺⁶ | 92.2 | | 5.81.10⁺⁷ | 97.3 | |
| | 3.3 | 1.60.10⁺⁸ | 34.9 | | 6.76.10⁺⁸ | 69.0 | |
| | 3.4 | 1.32.10⁺⁷ | 89.0 | | 6.38.10⁺⁷ | 97.1 | |
| 4-AR- | 4.1 | 3.85.10⁺⁷ | 68.0 | 69.4 ± 8.4 | 9.13.10⁺⁸ | 58.1 | 66.9 ± 7.8 |
| 100* | | | | | | | |
| 5 mg/kg/d | 4.2 | 2.60.10⁺⁷ | 78.4 | | 6.56.10⁺⁸ | 69.9 | |
| | 4.3 | 4.61.10⁺⁷ | 61.7 | | 5.94/10⁺⁸ | 72.8 | |
| 5-AR- | 5.1 | 1.72.10⁺⁷ | 85.7 | 72.0 ± 30.0 | 3.94.10⁺⁸ | 81.9 | 79.1 ± 20.9 |
| 100* | | | | | | | |
| 25 mg/kg/d | 5.2 | 8.94.10⁺⁶ | 92.6 | | 3.41.10⁺⁷ | 98.4 | |
| | 5.3 | 9.11.10⁺⁷ | 37.7 | | 9.38.10⁺⁸ | 90.8 | |
| 6-AR- | 6.1 | 5.17.10⁺⁷ | 57.1 | 71.7 ± 14.5 | 5.68.10⁺⁸ | 74.0 | 76.5 ± 13.3 |
| 100* | | | | | | | |
| 50 mg/kg/d | 6.2 | 3.38.10⁺⁷ | 71.9 | | 7.71.10⁺⁸ | 64.7 | |
| | 6.3 | 5.23.10⁺⁷ | 86.1 | | 2.00.10⁺⁸ | 90.8 | |
| 7-AR- | 7.1 | 1.59.10⁺⁷ | 86.8 | 66.5 ± 4.0 | 5.71.10⁺⁷ | 97.4 | 98.5 ± 0.9 |
| 100*/SMX | | | | | | | |
| 5/25 mg/kg/d | 7.2 | 5.31.10⁺⁷ | 55.9 | | 1.15.10⁺⁹ | 47.4 | |
| | 7.3 | 5.23.10⁺⁷ | 56.6 | | 4.70.10⁺⁸ | 78.4 | |
| 8-AR- | 8.1 | 9.55.10⁺⁶ | 92.1 | 94.0 ± 4.0 | 5.71.10⁺⁷ | 94.4 | 98.5 ± 0.9 |
| 100*/SMX | | | | | | | |
| 25/125/kg/d | 8.2 | 1.05.10⁺⁷ | 91.3 | | 1.89.10⁺⁷ | 99.1 | |
| | 8.3 | 1.69.10⁺⁶ | 98.6 | | 2.48.10⁺⁷ | 98.9 | |
| 9-AR- | 9.1 | 6.04.10⁺⁶ | 95.0 | 95.8 ± 1.2 | 1.44.10⁺⁷ | 99.3 | 98.1 ± 1.7 |
| 100*/SMX | | | | | | | |
| 50/250 mg/kg/d | 9.2 | 4.06.10⁺⁶ | 96.6 | | 6.76.10⁺⁷ | 96.9 | |
| 10- | 10.1 | 7.09.10⁺⁶ | 94.1 | 88.4 ± 5.8 | 3.69.10⁺⁷ | 98.3 | 96.8 ± 2.6 |
| Bactrim ® | | | | | | | |
| 50/250 mg/kg/d | 10.2 | 1.39.10⁺⁷ | 88.4 | | 1.37.10⁺⁸ | 93.7 | |
| | 10.3 | 2.09.10⁺⁷ | 82.6 | | 3.72.10⁺⁷ | 98.3 | |

*AR-100 = Iclaprim

### Example 4—Synergy Study of Iclaprim with Different Classes of Antibiotics

The in vitro synergistic potential of iclaprim was evaluated in “checkerboard” experiments using 31 different antibiotics against several strains of Staphylococcus aureus, Streptococcus pneumoniae, Haemophilus influenzae, Moraxella catarrhalis and Klebsiella pneumoniae. As discussed below, iclaprim showed potent activity against these pathogens with minimum inhibitory concentrations (MICs) ranging from 0.063 to 8 μg/ml, including with strains resistant to trimethoprim or trimethoprim-sulfamethoxazole. In terms of the synergistic potential, iclaprim was highly synergistic with the two sulfonamides tested (sulfamethoxazole and sulfadiazine). By contrast, iclaprim showed no synergy or antagonism with the other 29 antibiotics tested, including macrolides, aminoglycosides, quinolones, beta-lactams, trimethoprim, tetracyclines, rifampicin, and vancomycin.

Materials and Methods

Minimum inhibitory concentration (MIC) determination. Pathogens used were clinical isolates and type strains from the ATCC bacterial strain collection, including Staphylococcus aureus ATCC 25923, S. aureus 101, Streptococcus pneumoniae ATCC 49619, S. pneumoniae 1/1, Haemophilus influenzae ATCC 49766, Moraxella catarrhalis RA 21 and Klebsiella pneumoniae ATCC 33495. Resistance phenotypes per isolate are listed in Table 3.

In order to choose the appropriate concentrations to be tested in the checkerboard experiments, minimum inhibitory concentrations (MICs) were determined first. MIC determinations were performed under standard NCCLS conditions as described in Methods for dilution Antimicrobial susceptibility tests for bacteria that grow aerobically, Approved Standard-Sixth Edition (2003), NCCLS M7-A5, Vol. 23, No. 2, the entire disclosure of which is incorporated herein by reference, using doubling dilutions (0.125 to 128 μg/ml) of iclaprim and 31 antibiotics belonging to different classes (macrolides, aminoglycosides, lincosamides, quinolones, beta-lactams, folate pathway inhibitors such as trimethoprim and sulfonamides, tetracyclines, glycopeptides, fosfomycins, phenicols, ansamycins, fusidanes, coumarins, cyclic peptides; cf. Table 6) in microtiter plates. Stock solutions (10 mg/ml) of reference antibiotics were prepared in DMSO (except for tobramycin, gentamicin, lomefloxacin, tetracycline) and stored at 4° C. The bacteria were grown in Mueller-Hinton Broth (BBL Mueller-Hinton Broth II, cation adjusted). S. pneumoniae was grown in Mueller-Hinton Broth supplemented with 5% (v/v) lysed sheep blood. For the growth of H. influenzae HTM (Haemophilus test medium) was used containing Mueller-Hinton broth, yeast extract (5% w/v, Difco) and supplemented with NAD and hematin (HTM Supplement, Oxoid SR0158E). M. catarrhalis was grown in Brain Heart Infusion medium (Oxoid). The bacteria were incubated for 18 hours at 37° C. in ambient air except for S. pneumoniae, H. influenzae and M. catarrhalis, which were incubated in the presence of 5% CO2. The MIC was determined as the lowest concentration of an individual drug that lead to no visible growth.

3.1 Determination of the Synergistic Potential of Drugs In Vitro.

The synergistic potential of iclaprim against Gram-positive and Gram-negative bacteria was determined using the checkerboard assay as described in Eliopoulos GM and Moellering, Jr. RC (1991), Antimicrobial combinations, pp. 432-492, in V. Lorian (ed.), Antibiotics in laboratory medicine, 3rd Ed., The Williams & Wilkins Co., Baltimore, the entire disclosure of which is herein incorporated by reference, allowing multiple test concentrations of iclaprim to be assayed in the presence of various concentrations of the other antibiotic in microtiter plates. The same growth media and conditions were used as described in 3.2 below.

Two different dilutions were used for iclaprim depending on the MIC determined for the bacterium. If the MIC of iclaprim for a given organism was higher than 2 μg/ml, 11 dilutions ranging from 0.125 to 128 μg/ml were used, whereas in case of MICs lower than 2 μg/ml, 11 dilutions ranging from 0.002 to 2 μg/ml were applied. Seven multiple dilutions of the second antibiotic being tested in combination with iclaprim were applied in concentrations equal to 1) two to four concentrations above and 2) three to six concentrations below the MIC for that antibiotic of the bacterium tested. If the MIC was higher or equal to 16 μg/ml, 128 μg/ml was used as the highest concentration. For MICs between 0.125 and 8 μg/ml, the range tested started at a concentration 4 times higher than the MIC (e.g., if the MIC of iclaprim was 8 μg/ml, 7 dilutions ranging from 1 to 64 μg/ml were tested). Iclaprim and the other antibiotic being tested were also dispensed alone in the last row and in the last column, respectively, as controls.

The Fractional Inhibitory Concentrations (FIC) for each added agent were calculated and used to determine the sum of FIC (ΣFIC) indicative of the synergistic potential of a given combination as described in Veyssier P. (1999), Inhibiteurs de la dihydrofolate reductase, nitrohétérocycles (furanes) et 8-hydroxyquinoleines, pp. 995-1027, in A. Bryskier (ed.), Antibiotiques agents antibatériens et antifongiques, 1st Ed., Ellipses Edition Marketing SA, Paris, the entire disclosure of which is herein incorporated by reference. Synergy was defined whereby the ΣFIC was <0.5, indifference (no synergy nor antagonism) whereby ΣFIC was ≥0.5 but ≤4, and antagonism whereby ΣFIC was >4. Additivity as a special form of indifference was defined whereby the ΣFIC was ≥0.5 but <1, for example as described in Stevens, D L et al. (1998), In vitro antimicrobial effects of various combinations of penicillin and clindamycin against four strains of Streptococcus pyogenes, J. Antimicrob. Chemother. 42: 1266-1268, the entire disclosure of which is herein incorporated by reference. The overall calculation of ΣFIC was as follows:

$$FIC Index = \frac{MIC Drug A with Drug B}{MIC Drug A Alone} + \frac{MIC Drug B with Drug A}{MIC Drug B Alone}$$

3.2 Antimicrobial Activity of Iclaprim Against Gram-Positive and Gram-Negative Bacteria.

The resistance phenotypes of the bacterial strains according to the breakpoints published by the NCCLS Methods for dilution Antimicrobial susceptibility tests for bacteria that grow aerobically, Approved Standard-Sixth Edition (2003), NCCLS M7-A5, Vol. 23, No. 2 (supra) are shown in Table 3. All MIC values of iclaprim and the other antibiotics obtained are listed in Table 5 and Table 6. Iclaprim showed potent activity against the pathogens used in this study, with MICs ranging from 0.063 to 8 μg/ml. Iclaprim was also active against trimethoprim/trimethoprim-sulfamethoxazole-resistant strains of S. aureus (S. aureus 101) and S. pneumoniae (S. pneumoniae 1/1), having MICs of 1 μg/ml for both strains (see Table 5). (These strains were also cross-resistant to other antibiotics.) S. aureus 101 is resistant to trimethoprim, trimethoprim-sulfamethoxazole, penicillin, ampicillin, oxacillin, cefotaxime, gentamicin, tobramycin, erythromycin, and tetracycline. S. pneumoniae 1/1 is resistant to trimethoprim-sulfamethoxazole, penicillin, cefotaxime, and tetracycline (see Table 3). Iclaprim exhibited potent antibacterial activity against H. influenzae and M. catarrhalis, with MICs of 0.25 and 2 μg/ml, respectively, whereas iclaprim showed similar activity as compared with trimethoprim against K. pneumoniae with a MIC of 8 μg/ml (see Table 6).

3.3 Determination of the Synergistic Potential of Iclaprim In Vitro.

Iclaprim in combination with sulfamethoxazole exhibited synergism (ΣFIC ranging from 0.05 to 0.63) against both methicillin-susceptible and methicillin-resistant isolates of S. aureus, and penicillin-intermediate and penicillin-resistant isolates of S. pneumoniae (see Table 7). Notably, some of these isolates were also resistant to trimethoprim or trimethoprim-sulfamethoxazole (see Table 5). Iclaprim in combination with sulfamethoxazole also exhibited synergy against H. influenzae and M. catarrhalis (ΣFIC ranging from 0.09 to 0.56), whereas ΣFIC indicating additivity/indifference (ΣFIC 0.51-1.50) were found for the combination of iclaprim and sulfamethoxazole against K. pneumoniae ATCC 33495 (see Table 8). Sulfadiazine was also synergistic in combination with iclaprim against all the strains tested (ΣFIC 0.06-1.50), except for S. aureus 101 with ΣFIC indicating additivity/indifference (ΣFIC 0.75-1.13). No ΣFIC in the range indicating antagonism or synergy were observed with 29 antibiotics including macrolides, aminoglycosides, lincosamides, quinolones, beta-lactams, tetracyclines, glycopeptides, fosfomycins, phenicols, ansamycins, fusidanes, coumarins, cyclic peptides and trimethoprim. However, in combination with the two sulfonamides tested, namely sulfamethoxazole and sulfadiazine, FIC indices in the range indicating synergy were observed against the majority of isolates used (see Tables 5 and 6 below).

TABLE 3
Resistance phenotypes of bacterial strains used in this study. Listed are the
major clinically used drugs for which NCCLS breakpoints are defined.

| Strain | Resistance phenotype¹ |
|---|---|
| S. aureus 25923 | Susceptible to TMP, SXT, PEN, AMP, OXA, |
| | CTX, VAN, GEN, TOB, CLI, ERY, TET, CIP, |
| | RIF |
| S. aureus 101 | TMP^{R}, SXT^{R}, PEN^{R}, AMP^{R}, OXA^{R}, CTX^{R}, |
| | VAN^{S}, GEN^{R}, TOB^{R}, CLI^{S}, ERY^{R}, TET^{R}, CIP^{R}, |
| | RIF^{S} |
| S. pneumoniae 49619 | PEN¹; susceptible to SXT, CTX, VAN, CLI, |
| | ERY TET, RIF |
| S. pneumoniae 1/1 | PEN^{R}, SXT^{R}, CTX^{R}, VAN^{S}, CLI^{S}, ERY^{S}, TET^{R}, |
| | RIF^{S} |
| H. influenzae 49766 | Susceptible to SXT, AMP, CTX, TET, RIF |
| M. catarrhalis RA 21 | No published NCCLS breakpoints |
| K. pneumoniae 33495 | TMP^{S}, AMP^{R}, PIP^{I}, CTX^{S}, GEN^{S}, TOB^{S}, TET^{R} |

¹Abbreviations:
TMP, trimethoprim;
SXT, trimethoprim-sulfamethoxazole;
PEN, penicillin G;
AMP, ampicillin;
OXA, oxacillin;
PIP, piperacillin;
CTX, cefotaxime;
VAN, vancomycin;
GEN, gentamicin;
TOB, tobramycin;
CLI, clindamycin;
ERY, erythromycin;
TET, tetracycline;
RIF, rifampicin;
CIP, ciprofloxacin.

TABLE 4
Antimicrobial agents used for MIC determinations and synergy studies

| | Source | CatN |
|---|---|---|
| Iclaprim | Amcis | L991001 |
| Ampicillin | Fluka | 10047 |
| Bacitracin | Sigma | B-0125 |
| Cefotaxime | Fluka | 22128 |
| Cefsulodin sodium salt | Fluka | 22126 |
| Chloramphenicol | Fluka | 23275 |
| Clindamycin hydrochloride | Fluka | 27543 |
| Cloxacillin sodium salt | Fluka | 27555 |
| Doxycycline | Sigma | D 9891 |
| Erythromycin | Fluka | 45673 |
| Fusidic acid | Sigma | F-0881 |
| Gentamicin sulfate | Fluka | 48760 |
| Kanamycin sulfate | Fluka | 60615 |
| Lomefloxacin | Sigma | L 2906 |
| Moxalactam sodium salt | Fluka | 69962 |
| Norfloxacin | Sigma | MN 9890 |
| Novobiocin sodium salt | Fluka | 74675 |
| Oxacillin sodium salt | Fluka | 28221 |
| Penicillin G sodium salt | Fluka | 13752 |
| Fosfomycin | Fluka | 79492 |
| Piperacillin sodium salt | Fluka | 80624 |
| Puromycin dihydrochloride | Sigma | 82595 |
| Rifampicin | Fluka | 83907 |
| Roxithromycin | Sigma | R-4393 |
| Streptomycin sulfate | Sigma | 85880 |
| Sulfadiazine | Roche | — |
| Sulfamethoxazole | Sigma | S7507 |
| Tetracyclin | Fluka | 87128 |
| Thiostrepton | Fluka | 89053 |
| Tobramycin sulfate salt | Sigma | T 1783 |
| Trimethoprim | Fluka | F 92131 |
| Vancomycin | Fluka | 94747 |

TABLE 5
Antimicrobial activity of iclaprim and other antimicrobial
agents against Gram-positive bacteria. Minimum
inhibitory concentrations (MIC) are expressed in (μg/ml).

| | | | S. | S. |
|---|---|---|---|---|
| | S. aureus | S. aureus | pneumoniae | pneumoniae |
| Antibiotic | 25923 | 101 | 49619 | 1/1 |
| Iclaprim | 0.063 | 1 | 0.125 | 1 |
| Trimethoprim | 1 | >128 | 8 | 128 |
| Penicillin G | 0.031 | 128 | 0.25 | 4 |
| Cloxacillin | 0.25 | 2 | 8 | 64 |
| Ampicillin | 0.5 | >128 | 0.5 | 16 |
| Oxacillin | 0.125 | 8 | 2 | 32 |
| Piperacillin | 0.5 | >128 | 2 | 8 |
| Cefotaxime | 4 | 32 | 0.25 | 8 |
| Cefsulodine | 64 | 64 | 16 | >128 |
| Moxalactam | 1 | 4 | 1 | 4 |
| Vancomycin | 2 | 2 | 0.5 | 0.5 |
| Bacitracin | >128 | 32 | 32 | 8 |
| Fosfomycin | 8 | 64 | 32 | 32 |
| Gentamicin | 0.5 | >128 | 32 | 32 |
| Kanamycin | 2 | >128 | 32 | 128 |
| Tobramycin | 0.5 | >128 | 32 | 32 |
| Streptomycin | 4 | 8 | 32 | >128 |
| Puromycin | 32 | 16 | 8 | 8 |
| Clindamycin | 0.25 | 0.25 | 0.125 | 0.125 |
| Erythromycin | 0.5 | >128 | 0.125 | 0.125 |
| Roxithromycin | 1 | >128 | 0.5 | 0.5 |
| Chloramphenicol | 8 | 64 | 4 | 16 |
| Fusidic acid | 0.25 | 0.125 | 4 | 8 |
| Tetracycline | 0.5 | 128 | 0.25 | 16 |
| Thiostrepton | 0.5 | 0.5 | 0.063 | 0.063 |
| Doxycycline | 0.5 | 16 | 0.5 | 2 |
| Lomefloxacin | 1 | 128 | 8 | 8 |
| Norfloxacin | 1 | 128 | 4 | 4 |
| Rifampicin | 0.031 | 0.031 | 0.031 | 0.063 |
| Novobiocin | 0.5 | 0.25 | 2 | 4 |
| Sulfadiazine | >128 | >128 | >128 | >128 |
| Sulfamethoxazole | >128 | >128 | >128 | >128 |
| Trimethoprim- | 0.063/1.19 | 16/304 | 0.5/9.5 | 8/152 |
| sulfamethoxazole¹ | | | | |

¹Testing was carried out using a 1:19 ratio of trimethoprim/sulfamethoxazole

TABLE 6
Antimicrobial activity of iclaprim and other antimicrobial agents
against Gram-negative bacteria. Minimum inhibitory
concentrations (MIC) are expressed in (μg/ml).

| | H. Influenza | M. catarrhalis | K. pneumonia |
|---|---|---|---|
| Antibiotic | 49766 | RA 21 | 33495 |
| Iclaprim | 0.25 | 2 | 8 |
| Trimethoprim | 0.5 | 64 | 8 |
| Penicillin G | 0.25 | 16 | 128 |
| Cloxacillin | 16 | 64 | >128 |
| Ampicillin | 1 | 16 | >128 |
| Oxacillin | 16 | 32 | >128 |
| Piperacillin | 0.031 | 0.25 | 32 |
| Cefotaxime | 0.063 | 4 | 0.5 |
| Cefsulodine | >128 | >128 | >128 |
| Moxalactam | 1 | 2 | 8 |
| Vancomycin | 128 | >128 | >128 |
| Bacitracin | >128 | 8 | >128 |
| Fosfomycin | 0.062 | 128 | >128 |
| Gentamicin | 2 | 2 | 1 |
| Kanamycin | 2 | 4 | 4 |
| Tobramycin | 0.5 | 4 | 1 |
| Streptomycin | 0.5 | 8 | 64 |
| Puromycin | 8 | 4 | >128 |
| Clindamycin | 8 | 4 | >128 |
| Erythromycin | 8 | 0.125 | 128 |
| Roxithromycin | 16 | 0.25 | >128 |
| Chloramphenicol | 0.5 | 0.5 | >128 |
| Fusidic acid | 1 | <0.008 | >128 |
| Tetracycline | 0.5 | 0.25 | 128 |
| Thiostrepton | 32 | 0.5 | >128 |
| Doxycycline | 0.5 | 0.5 | >128 |
| Lomefloxacin | 0.125 | 0.5 | 4 |
| Norfloxacin | 0.125 | 0.5 | 2 |
| Rifampicin | 0.063 | 0.063 | >128 |
| Novobiocin | 0.25 | 0.25 | 128 |
| Sulfadiazine | >128 | >128 | >128 |
| Sulfamethoxazole | 128 | >128 | >128 |
| Trimethoprim- | ≤0.031/0.59 | ND² | ND |
| sulfamethoxazole¹ | | | |

¹Testing was carried out using a 1:19 ratio of trimethoprim/sulfamethoxazole
²ND, not determined

TABLE 7
Synergistic potential of iclaprim against Gram-positive bacteria.
The ΣFIC indicates the synergistic potential of a given combination.

| | S. aureus | | S. aureus | | S. pneumoniae | | S. pneumoniae | |
|---|---|---|---|---|---|---|---|---|
| | 25923 | | 101 | | 49619 | | 1/1 | |
| Antibiotics¹ | Σ FIC | Con² | Σ FIC | Con | Σ FIC | Con | Σ FIC | Con |
| Trimethoprim | 1.00-1.25 | Ind | 1.01-1.50 | Ind | 1.00-1.25 | Ind | 0.56-1.03 | Ad |
| Penicillin G | 1.00-1.25 | Ind | 1.00-1.25 | Ind | 1.00-1.25 | Ind | 0.75-1.13 | Ad |
| Cloxacillin | 0.99-1.25 | Ad | 1.00-1.25 | Ind | 1.00-1.25 | Ind | 1.00-1.25 | Ind |
| Ampicillin | 1.00-1.00 | Ind | 1.01-1.50 | Ind | 1.00-1.25 | Ind | 1.00-1.25 | Ind |
| Oxacillin | 1.06-1.50 | Ind | 1.00-1.25 | Ind | 1.06-1.50 | Ind | 1.06-1.50 | Ind |
| Piperacillin | 1.06-1.50 | Ind | 1.01-1.50 | Ind | 1.02-1.50 | Ind | 0.63-1.13 | Ad |
| Cefotaxime | 1.03-1.50 | Ind | 1.06-1.50 | Ind | 1.00-1.25 | Ind | 0.63-1.06 | Ad |
| Cefsulodine | 1.00-1.25 | Ind | 1.00-1.25 | Ind | 1.13-1.50 | Ind | 1.01-1.50 | Ind |
| Moxalactam | 0.56-1.25 | Ad | 0.63-1.25 | Ad | 1.00-1.25 | Ind | 1.13-1.50 | Ind |
| Vancomycin | 1.03-1.50 | Ind | 0.75-1.25 | Ad | 1.00-1.25 | Ind | 0.56-1.03 | Ad |
| Bacitracin | 0.74-1.13 | Ad | 0.75-1.13 | Ad | 0.75-1.13 | Ad | 0.51-1.06 | Ad |
| Fosfomycin | 1.13-1.50 | Ind | 1.03-1.50 | Ind | 1.06-1.50 | Ind | 1.00-1.06 | Ind |
| Gentamicin | 2.05-2.48 | Ind | 1.01-1.50 | Ind | 0.75-1.13 | Ad | 1.06-1.50 | Ind |
| Kanamycin | 1.06-1.50 | Ind | 1.01-1.50 | Ind | 1.00-1.25 | Ind | 1.02-1.50 | Ind |
| Tobramycin | 0.99-1.25 | Ad | 1.01-1.50 | Ind | 1.00-1.25 | Ind | 0.63-1.13 | Ad |
| Streptomycin | 1.13-1.50 | Ind | 1.13-1.50 | Ind | 1.00-1.25 | Ind | 1.01-1.50 | Ind |
| Puromycin | 1.06-1.50 | Ind | 1.13-1.50 | Ind | 0.63-1.25 | Ad | 1.13-1.50 | Ind |
| Clindamycin | 2.02-2.48 | Ind | 2.03-2.50 | Ind | 1.00-1.25 | Ind | 0.63-1.06 | Ad |
| Erythromycin | 1.13-1.50 | Ind | 1.01-1.50 | Ind | 1.06-1.50 | Ind | 1.06-1.50 | Ind |
| Roxithromycin | 1.06-1.50 | Ind | 1.01-1.80 | Ind | 1.02-1.50 | Ind | 0.75-1.13 | Ad |
| Chloramphenicol | 1.13-2.50 | Ind | 0.63-1.13 | Ad | 0.75-1.25 | Ad | 1.13-0.75 | Ind |
| Fusidic acid | 2.03-2.50 | Ind | 1.06-1.50 | Ind | 1.13-1.50 | Ind | 1.06-1.50 | Ind |
| Tetracycline | 2.05-2.48 | Ind | 1.02-2.50 | Ind | 0.75-1.25 | Ad | 0.75-1.13 | Ad |
| Thiostrepton | 1.02-1.50 | Ind | 1.50-2.25 | Ind | 1.12-1.50 | Ind | 1.12-1.50 | Ind |
| Doxycycline | 1.02-1.50 | Ind | 1.06-1.50 | Ind | 1.02-1.51 | Ind | 1.25-1.50 | Ind |
| Lomefloxacin | 1.06-1.50 | Ind | 1.02-1.50 | Ind | 0.75-1.13 | Ad | 0.75-1.13 | Ad |
| Norfloxacin | 0.56-1.25 | Ad | 1.02-1.50 | Ad | 1.00-1.25 | Ind | 1.13-1.50 | Ind |
| Rifampicin | 0.54-1.25 | Ad | 0.75-1.25 | Ad | 0.75-0.76 | Ad | 0.56-1.12 | Ad- |
| Novobiocin | 1.02-1.50 | Ind | 1.03-1.50 | Ind | 0.75-1.13 | Ad | 1.03-1.50 | Ind |
| Sulfadiazine | 0.06-0.53 | Syn | 0.75-1.13 | Ad | 0.13-0.53 | Syn | 0.28-0.56 | Syn |
| Sulfamethoxazole | 0.06-0.53 | Syn | 0.19-0.63 | Syn | 0.05-0.52 | Syn | 0.16-0.63 | Syn |

¹Antibiotics tested in combination with iclaprim
²“Conclusion”: Synergy (Syn); Additivity (Ad); Indifference (Ind).

TABLE 8
Synergistic potential of iclaprim against Gram-negative bacteria.
The ΣFIC indicates the synergistic potential of a given combination.

| | H. influenzae | | M. catarrhalis | | K. pneumoniae | |
|---|---|---|---|---|---|---|
| | 49766 | | RA 21 | | 33495 | |
| Antibiotics¹ | Σ FIC | Con² | Σ FIC | Con | Σ FIC | Con |
| Trimethoprim | 1.00-1.25 | Ind | 0.75-1.13 | Ad | 1.13-1.50 | Ind |
| Penicillin G | 1.03-1.50 | Ind | 1.13-1.50 | Ind | 0.52-1.25 | Ad |
| Cloxacillin | 1.13-1.50 | Ind | 0.53-1.00 | Ad | 1.01-2.50 | Ind |
| Ampicillin | 1.13-1.50 | Ind | 1.00-1.25 | Ind | 0.52-1.01 | Ad |
| Oxacillin | 1.00-1.25 | Ind | 1.06-1.50 | Ind | 1.01-1.50 | Ind |
| Piperacillin | 0.54-1.25 | Ad | 1.03-1.50 | Ind | 0.53-1.25 | Ad |
| Cefotaxime | 0.75-1.25 | Ad | 0.56-1.03 | Ad | 1.13-1.50 | Ind |
| Cefsulodine | 1.01-1.50 | Ind | 1.00-1.25 | Ind | 1.01-1.50 | Ind |
| Moxalactam | 1.06-1.50 | Ind | 1.00-1.25 | Ind | 0.75-1.25 | Ad |
| Vancomycin | 1.00-1.25 | Ind | 0.75-1.13 | Ad | 1.01-1.50 | Ind |
| Bacitracin | 1.01-1.50 | Ind | 0.56-1.25 | Ad | 1.01-1.50 | Ind |
| Fosfomycin | 0.74-1.12 | Ad | 1.02-1.50 | Ind | 1.01-1.50 | Ind |
| Gentamicin | 1.02-1.13 | Ind | 1.00-1.25 | Ind | 0.63-1.25 | Ad |
| Kanamycin | 1.02-1.13 | Ind | 1.00-1.25 | Ind | 0.63-0.75 | Ad |
| Tobramycin | 1.02-1.50 | Ind | 0.63-1.25 | Ad | 0.52-1.13 | Ad |
| Streptomycin | 1.02-1.50 | Ind | 1.06-1.50 | Ind | 0.63-1.06 | Ad |
| Puromycin | 1.06-1.50 | Ind | 1.06-1.50 | Ind | 1.01-1.50 | Ind |
| Clindamycin | 1.13-1.50 | Ind | 1.00-1.25 | Ind | 1.01-1.50 | Ind |
| Erythromycin | 0.75-1.00 | Ad | 1.06-1.50 | Ind | 0.56-1.25 | Ad |
| Roxithromycin | 1.03-1.50 | Ind | 1.03-1.50 | Ind | 1.01-1.50 | Ind |
| Chloramphen- | 1.06-1.50 | Ind | 1.25-1.50 | Ind | 1.01-1.50 | Ind |
| icol | | | | | | |
| Fusidic acid | 1.06-1.50 | Ind | 1.00-1.00 | Ind | 1.00-1.25 | Ind |
| Tetracycline | 0.56-1.25 | Ad | 1.13-1.50 | Ind | 0.56-1.03 | Ad |
| Thiostrepton | 1.02-1.50 | Ind | 1.02-1.50 | Ind | 1.01-1.50 | Ind |
| Doxycycline | 1.00-1.00 | Ind | 1.02-1.50 | Ind | 1.01-1.50 | Ind |
| Lomefloxacin | 1.06-1.50 | Ind | 0.63-1.13 | Ad | 1.13-1.50 | Ind |
| Norfloxacin | 0.52-1.25 | Ad | 0.56-1.13 | Ad | 0.75-1.13 | Ad |
| Rifampicin | 1.12-1.50 | Ind | 0.75-1.12 | Ad | 1.01-1.50 | Ind |
| Novobiocin | 1.03-1.50 | Ind | 1.00-1.05 | Ind | 1.00-1.25 | Ind |
| Sulfadiazine | 0.16-0.53 | Syn | 0.09-0.50 | Syn | 0.27-1.50 | Syn |
| Sulfamethox- | 0.09-0.52 | Syn | 0.09-0.56 | Syn | 0.51-1.50 | Ad |
| azole | | | | | | |

¹Antibiotics tested in combination with iclaprim
²“Conclusion”: Synergy (Syn); Additivity (Ad); Indifference (Ind).

### Example 5—Study of Iclaprim with Sulfonamide Antibiotics

Particularly suitable combinations of iclaprim with sulfonamide antibiotics will be identified by spectrum screening and checkerboard synergy tests. Tests would be performed to determine the relevant spectrum of activity and potential areas of synergy for iclaprim with sulfonamide antibiotics, in comparison to TMP/SMZ, and standard of care treatments for pathogens in cSSI, RTI, UTI and GI, using a Tier 1 panel of organisms.

Candidate sulfonamide antibiotics will be selected based on PK/PD and tolerability/safety data, and assayed for antimicrobial spectrum against the Tier 1 panel. Checkerboard combination tests will be performed on the relevant diaminopyrimidine/sulfonamide combinations.

### Example 6—Evaluation of Iclaprim with Sulfonamide Antibiotics

Following identification and selection of candidate combination(s), deeper testing of spectrum and coverage of pathogens will be done with larger panels of organisms (Tier 2). Bacteriostatic vs. bactericidal activity will be determined for the candidate combination(s) against select organisms and different medium, reflective of in vivo conditions at sites of infection.

### Example 7—Selection of Iclaprim and Sulfonamide Antibiotic Combination

PAE and resistance studies would be performed following PK studies and PD analysis. An ideal candidate will cover the spectrum of pathogens for the therapeutic indications targeted, will exhibit synergy in antibacterial activity, cidality, low selection of antibiotic resistance (AMR; both spontaneous mutational frequency, and resistance development via passaging), and post-antibiotic effect (PAE) against key pathogens, and a PK/PD profile suitable for the distribution of drugs at the optimal ratio to sites of infection. Microbiology studies to be performed include:
The post-antibiotic effect (PAE) of the candidate combination(s) will be determined for key pathogens under commonly utilized methods from the literature.
The mutation frequency to the combination and its components will be determined for key pathogens by the broth dilution and plating methods. Mutant selection studies may also be performed.

Materials and Methods

MIC Protocol:

Details of the MIC assay are briefly described below.

### Procedures:

### Equipment:

McFarland standard 0.5.
Turbidity meter.
Sterile 96-well U-bottom polystyrene assay plates with lids.
Disposable sterile microbiological loops (1 μl and 10 μl).
Multichannel pipette.
Microplate reader with mirror OR manual reading.
Disposable reservoir for reagents.
Graduated pipettes (20 μl-1000 μl).
Sterile pipette tips.

### Media:

Sterile normal saline, 4 ml volumes in tubes for turbidity measurement.
Cation adjusted Mueller-Hinton II broth or otherwise, as per CLSI guideline.
Trypticase soy agar plates for purity control of inoculum suspensions.

### Bacterial Strains:

Gram-Negative Pathogens, including Escherichia coli, Klebsiella pneumoniae, Enterobacter sp., Salmonella sp., Haemophilus influenza, Serratia marcescnes Moraxella catarrhalis and other pathogens as required
Gram-Positive Pathogens, including Staphylococcus aureus, Enterococcus faecalis, Enterococcus faecium, Streptococcus pneumoniae, and other pathogens as required
Quality Control Strains: P. aeruginosa ATCC 27853, E. faecalis, ATCC 29212, S. aureus ATCC 29213 and E. coli ATCC 25922.

### Experimental Antibiotics:

Iclaprim, trimethoprim, TMP-SMX and several sulfonamide antibiotics, including sulfamethoxazole.

### Control Antibiotics:

A set of standard-of-care antibiotics.

### Preparation of Antibiotic Dilutions:

Testing will cover approximately 10-12 serial doubling dilutions.
Test Substance and antibiotics will be prepared.

### Standardization of Inoculum:

From a pure O/N culture, material will be picked from at least 3-4 colonies. Material will be suspended in 4 ml saline in tubes.
Adjust to McFarland 0.5 (turbidity meter).
If a turbidity meter is not available: Compare visually with the McFarland 0.5 standard using white paper with black lines as background.
The McFarland 0.5 suspension is diluted as follows for the species tested.
Gram-neg.: 10 μl McFarland 0.5 into 10 ml broth.
Gram-pos.: 50 μl McFarland 0.5 into 10 ml broth.
Mix. The suspensions are used for inoculation within 15 minutes.

### Inoculation and Incubation:

The diluted antibiotics (2-fold final assay concentration; 100 μl) in microtiter plate wells are over-layered with 100 μl of the inoculum suspension in cation adjusted Mueller Hinton Broth using a multi-channel pipette.
Lids are placed on the inoculated plates and incubated at 37° C. for 18-22 hours.

### Purity Control:

Spread 10 μl of the inoculation-suspension on a Trypticase soy agar plate. Incubate at 35° C. overnight.

### Reading MIC/Interpretation of Results:

Read plates either manually or using Plate reader as follows:
Use the record sheet for orientation of the plates.
Check growth in the 3 positive control wells.
The MIC is read as the lowest concentration without visible growth.

### Quality Control Ranges are Observed and Recorded:

The readings are compared with the table of MIC standard given by the CLSI.

Cidality and PAE Protocols:

CLSI guidelines for “Determining the Bactericidal Activity of Antimicrobial Agents” (M26-A) will be generally followed, to determine minimal bactericidal concentrations (MBCs), and growth/viability measurements post sub-MIC treatment to determine post-antibiotic effect (PAE), with variations in the test determined for each study performed, in study-specific protocols.

Resistance Frequency Protocols:

Spontaneous mutational frequency will be determined by plating a quantified inoculum on Mueller-Hinton Agar containing agreed upon fold of MIC followed by confirmation of resistant colonies by broth microdilution assay; and calculated as the proportion of resistant bacterial cells versus entire plated population prior to antibiotic exposure. Resistance by passaging will be conducted according to commonly utilized method from the literature.

While the present disclosure has been discussed in terms of certain embodiments, it should be appreciated that the present disclosure is not so limited. The embodiments are explained herein by way of example, and there are numerous modifications, variations and other embodiments that can be employed that would still be within the scope of the present disclosure.

1. A pharmaceutical formulation comprising iclaprim or an enantiomer thereof and a sulfonamide antibiotic.

2. The formulation of claim 1, wherein the sulfonamide antibiotic is selected from the group consisting of sulfisoxazole, sulfadimethoxine, sulfamethoxazole, 4-sulfanilamido-5,6-dimethoxy-pyrimidine (sulfadoxine), 2-sulfanilamido-4,5-dimethyl-pyrimidine, sulfaquinoxaline, sulfadiazine, sulfamonomethoxine, and 2-sulfanilamido-4,5-dimethyl-isoxazole or dapsone.

3. The formulation of claim 1, wherein the sulfonamide antibiotic is sulfamethoxazole.

4. The formulation of claim 1, wherein the iclaprim or an enantiomer thereof and sulfonamide antibiotic are present in a ratio of from about 1:0.1 to 1:4.5 of iclaprim or an enantiomer thereof to sulfonamide antibiotic.

5. The formulation of claim 1, wherein the iclaprim or an enantiomer thereof and sulfonamide antibiotic are present in a ratio of from about 1:1, 1:2 or 1:3 of iclaprim or an enantiomer thereof to sulfonamide antibiotic.

6. The formulation of claim 1, wherein the iclaprim or an enantiomer thereof and sulfonamide antibiotic are present in a ratio of from about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4 and 1:4.5 of iclaprim or an enantiomer thereof to sulfonamide antibiotic.

7. The formulation of claim 1, which is formulated for intravascular administration.

8. The formulation of claim 7, wherein the intravascular administration is intravenous.

9. The formulation of claim 1, which is formulated for oral administration.

10. The formulation of claim 9, wherein the formulation comprises a tablet, caplet or capsule.

11. The formulation of claim 10, wherein the tablet further comprises a surfactant, a lubricant, a disintegrant, a diluent or a binder.

12. The formulation of claim 10, wherein the tablet further comprises docusate sodium, sodium benzoate, sodium starch glycolate, magnesium stearate and pregelatinized starch.

13. The formulation of claim 1, comprising about 160 mg of iclaprim or an enantiomer thereof and about 160 to 480 mg of sulfonamide antibiotic.

14. The formulation of claim 1, comprising about 160 mg of iclaprim or an enantiomer thereof and about 160 mg of sulfonamide antibiotic; about 160 mg of iclaprim or an enantiomer thereof and about 320 mg of sulfonamide antibiotic; or about 160 mg of iclaprim or an enantiomer thereof and about 480 mg of sulfonamide antibiotic.

15. The formulation of claim 1, wherein the iclaprim or an enantiomer thereof is substantially the R-enantiomer of iclaprim.

16. A method of treating a bacterial or fungal infection in a subject, comprising administering to the subject a therapeutically effective amount of a pharmaceutical formulation comprising iclaprim or an enantiomer thereof and a sulfonamide antibiotic.

17. The method of claim 16, wherein the sulfonamide antibiotic is selected from the group consisting of sulfisoxazole, sulfadimethoxine, sulfamethoxazole, 4-sulfanilamido-5,6-dimethoxy-pyrimidine (sulfadoxine), 2-sulfanilamido-4,5-dimethyl-pyrimidine, sulfaquinoxaline, sulfadiazine, sulfamonomethoxine, and 2-sulfanilamido-4,5-dimethyl-isoxazole or dapsone.

18. The method of claim 16, wherein the sulfonamide antibiotic is sulfamethoxazole.

19. The method of claim 16, wherein the iclaprim or an enantiomer thereof and sulfonamide antibiotic are present in the pharmaceutical formulation in a ratio of from about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4 and 1:4.5 of iclaprim or an enantiomer thereof to sulfonamide antibiotic.

20. The method of claim 16, wherein the bacterial or fungal infection comprises infection with Streptococcus pneumoniae; Escherichia coli; Haemophilus influenzae; Morganella morganii; Proteus mirabilis; Proteus vulgaris; Shigella flexneri; Shigella sonnei; Pneumocystis jiroveci; or methicillin-resistant Staphylococcus aureus.
